(19) United States
(12) Patent Application Publication
(10) Pub. No.: US 2019/0015551 A1
(43) Pub. Date: Jan. 17, 2019

(54) CONSTRUCT FOR PREVENTING IMMUNOLOGICAL REJECTION GENERATED WHEN USED IN TRANSPLANTS, AND METHOD FOR USING COLLAGEN IN A GEL STATE, IN THE FORM OF DRY LYOPHILISED SPONGY MOULDINGS AND 3D MATRICES

(71) Applicant: ADG I+D SPA, Santiago (CL)

(72) Inventors: Ruby Patricia VALDIVIA ALCOTA, Santiago (CL); Motoe KATO, Santiago (CL)

(21) Appl. No.: 16/067,301

(22) PCT Filed: Dec. 29, 2016
(86) PCT No.: PCT/IB2016/058084

§ 371 Date: Jun. 29, 2018

(30) Foreign Application Priority Data
Dec. 30, 2015 (CL) 2015-03793

(51) Int. Cl. A61L 27/36; A61L 27/24; C12N 5/074; C12N 5/071

(52) U.S. Cl. CPC A61L 27/3604; A61L 27/24; A61L 27/3641; A61L 27/60; C12N 5/0696; C12N 5/0692; A61L 27/3683

(57) ABSTRACT

The present invention relates to a construct for preventing immunological rejection when used in transplant, whether xenotransplant or allotransplant, which comprises: a) cells with regenerative capacity; b) collagen in 3-D gel state, or also called collagen 3-D matrix, obtained from pig's head cheeks; c) culture medium; d) an element aimed to be transplanted; and e) dry lyophilized spongy collagen molded with the shape of the element to be transplanted; It also relates to a method for producing collagen in a gel state in the form of dry lyophilized spongy mold and 3-D matrix.

### FIELD OF THE INVENTION

The present invention relates to the biomedical industry, tissue engineering and tooth regeneration. In particular, the present invention relates to a construct for preventing immunological rejection when used in transplant in the treatment of diseases (without the use of immunosuppressive drugs), whether xenotransplant or allotransplant. The present invention also relates to the method for obtaining said construct. Additionally, it is described a method for producing collagen in the gel state, either in 3-D form or as dry lyophilized spongy collagen molds with the shape of the element to be transplanted. Finally, the present invention relates to the use of the construct for preventing the immunological rejection produced during the transplantation thereof.

### STATE OF ART

The dental tissue regeneration procedures have their origin since around 1952, when the regeneration of pulpal cells was reported by the application of calcium hydroxide in a clinical case of amputation of a vital pulp. From that evidence numerous advances were made using local stimuli during the endodontics always depending on healthy pre-existing tissue; however, the nature of these partial regenerations was unknown until it was discovered that there were adult mesenchymal stem cells in the tooth pulp. The pulp of permanent teeth contains stem cells (Human Dental Pulp Stem Cells, DPSCs), which form different types of clonogenic colonies in cultures with greater frequency (22 to 70 colonies/104 seeded cells) and greater cell proliferation (72% vs. 42%) that a similar number of cells seeded from bone marrow, but in less proportion than the pulp of primary exfoliated teeth of children from 7 to 8 years of age (Stem cells from human exfoliated deciduous teeth, SHED); when they are maintained for a long time in cultures with osteogenic medium the DPSCs secrete mineralizing matrix (mineralization nodes) demonstrating high levels of calcium, however in cultures it was not possible to demonstrate the presence of dentin due to the nature of the in vitro culture. These stem cells, when transplanted in the presence of hydroxyapatite and tricalcium phosphate (HA/TCP) in the subcutaneous dorsum of immunosuppressed mice, showed the formation of a pulp/dentin complex, with blood vessels, dentin secretion and ordered odontoblasts. In addition to their dentinogenic potential, stem cells derived from adult dental pulp have adipogenic and neurogenic, chondrogenic and myogenic potential, confirming the plasticity and heterogeneity of this population of mesenchymal stem cells in vitro. However, its use in vivo has not been demonstrated in the absence of immunosuppression for preventing its immunological rejection. When comparing the SHED with the DPSC, it is concluded that the primary teeth of children are a source of stem cells very different from the stem cells present in the pulp of permanent teeth, the neurogenic potential of the SHED makes them very interesting for neuronal regeneration and even more because it was possible to isolate the cells with characteristics of embryonic stem cells (ES-cells) from these tissues; however, this last characteristic could constitute a risk of malignancy due to the infinite potential of multiplications that the embryonic stem cells possess. On the other hand, SHEDs are not a good source of tissue to regenerate a dental germ because they do not form the pulp/dentin complex and are only bone inducers, because they do not differentiate into osteoblasts.

The third type of stem cells of dental tissue is the SCAP (Stem Cells from Apical Papilla), obtained from the apical third molar papilla included in early steps of development (Nolla, C-D-E); which could be considered the source of the cells of the radicular pulp (odontoblasts producing coronal dentin) and it is tempting to think that these same SCAPs give rise to DPSCs, but it has not been demonstrated that SCAPs constitute DPSCs in the future pulp of the adult tooth and several differences have been demonstrated between these two types of MSCs. The transplant of SCAP colonies in vivo, as was done with the DPSCs in immunosuppressed mice, showed that they form the pulp/dentin complex, which gives them advantages due to their development potential; on the other hand, another type of studies in which the apical papilla was extracted from developing teeth, showed that this tissue has an important role in the formation of the root because although the pulp remained intact, the germ without the papilla was not able to form root. Thus, it is concluded that in the dental germ the SCAP cells are fundamental for the development of the dental root.

The fourth dental tissue in which a group of MSCs was characterized is the dental follicle; which can be obtained from third molars including humans in early development, separating the ectomesenchyme surrounding the enamel organ in the dental papilla. This tissue contains clonogenic stem cells (Dental Follicle Precursor Cells, DFPCs) that in culture show multipotentiality myogenic, neurogenic, adipogenic, chondrogenic, in addition to the cementogenic and odontogenic potential; those that, transplanted in vivo, give rise to fibrous tissue similar to the periodontal ligament, but do not form dentine, cement, or bone. On the other hand, immortalized cells of the dental follicle are able to form the complete periodontal ligament when they are transplanted in vivo, however the complexity of this immortalization technique makes them difficult to use clinically.

From the background described above, it is clear that the regeneration of the dental organ requires more than one type of stem cell due to its epithelial-mesenchymal origin and despite recent advances in the properties of stem cells, a biological solution is still lacking which allow to anchor the tooth to the alveolar niche and return the functionality to the masticatory system of the individual from a type of stem cells. The periodontal ligament contains cells capable of differentiating into cementoblasts and osteoblasts; however, it also contains a reservoir of stem cells with the characteristics of MSCs called Periodontal Ligament Stem Cells (PDLSCs), these cells isolated in culture are characterized by responding to induction with the corresponding means to differentiate adipocytes, chondrocytes and osteoblasts. In addition, its ability to differentiate neuronal precursors was demonstrated. The transplant of PDLSCs in immunosuppressed mice showed the formation of periodontal ligament containing Sharpey fibers attached to the cement formed by these cells similar to what was observed under physiological conditions in an individual; moreover, the transplant of these cells to the periodontal region of rats with surgically induced damage showed that the periodontal tissue formed adhered to the alveolar surface and the tooth surface. To date, all publications indicate that dental stem cell transplants have been performed with animal immunosuppression and only using a rat or pig reparative model. The present invention shows a construct that can contain adult periodontal ligament stem cells from teeth extracted in dental clinics and that prevents immunological rejection when used in the treatment of diseases or conditions related to human teeth, without the use of immunosuppression.

The attempt to regenerate a complete tooth was initiated by research groups experimenting through heterotypic recombination of dental tissues that has the complexity of requiring the formation of four different tissue types: pulp, dentin, cement (these last two of mesenchymal origin), and finally, enamel, mineralized tissue of epithelial origin, the hardest of the human body. This enamel is secreted by the ameloblasts that exist only during development, once the tooth erupts the ameloblasts die. The mineralization process takes more than six years, from the sixth week of intrauterine life to the childhood in which the replacement of the primary teeth occurs, which do not have dental root, by the definitive teeth that if they are not damaged will remain in the mouth until the death of the individual due to they have dental roots. These mineralized tissues, are joined from the root to the alveolar bone niche by the periodontal ligament, which must also be regenerated if lost due to pathological or traumatic reasons. However, this regeneration of the periodontal ligament will only take a couple of months compared to the years it would normally take to regenerate the mineralized tissues. Without the discovery of stem cells, dentistry focused on replacing the dental root to recover the function of occlusion using artificial prostheses called dental implants.

Dental implants currently existing in markets are composed of a metal base, usually titanium.

These metals are implanted in the alveolar bone without being able to regenerate the periodontal complex, so they do not have a natural relationship due to the lack of dissipation and transmission of forces, lack of proprioception and lack of stereognosis. These are properties that only have the natural or biological tooth, because only the cells can be structured to form blood vessels, innervations and extracellular matrix rich in fibers besides producing nutrients, chemical and electrical signals that inform the brain of proprioception, volumetric three-dimensional dimension, transmission and dispersion of masticatory forces that ultimately help maintain the homeostasis of the alveolar bone and the life signal of the tooth that prolongs the competitive age of the individual and prevents premature aging.

On the other hand, titanium implants are susceptible to loosening or producing infections such as gingivitis or periodontitis, due to the unnatural relationship between the bone and the metallic implant.

The world market for metallic dental implants for 2010 was approximately 3,200 million dollars. By 2015 there was an annual growth rate of 6%, reaching a value of 4,200 million dollars. If it is considered that the Latin American market is 10%, it is a potential market of 420 million dollars for the year 2015.

Therefore, it is fundamental to develop a product that allows the implant or development of dental pieces of biological basis for dental regeneration, that prevent premature aging of dental tissues, that provide greater well-being and life quality, and having a greater biocompatibility, at the same time reducing the problems generated by the unnatural relationship between the bone and the metallic implant. In view of this problem, the present application presents a collagen-containing construct that serves as a support for the implantation of biological-based pieces, which appears as an alternative to currently used implants, and which also improves biocompatibility by drastically reducing rejection levels to practically zero. Thus, the present application comprises products such as a construct for preventing immune rejection produced when used in transplant in the treatment of diseases (without the use of immunosuppressive drugs), either xenotransplant or allotransplant, a method for obtaining this construct, a method for producing collagen in a gel state, either in the form 3-D or as dry lyophilized spongy collagen molds with the shape of the element to be transplanted, and the use thereof.

In the prior art there is the publication US 20100196854 which describes a method for reconstructing teeth using a carrier of a biocompatible material that may include hydroxyapatite loaded with Stem cells of apical papilla (SCAPs), Periodontal Ligament Stem Cells (PDLSC) and Dental Pulp Stem Cells (DPSC), which could be used especially as a root reconstructor. However this document does not refer to the fact that the biocompatible material is collagen nor the specific steps and components necessary to produce the construct of the present invention. Therefore, it does neither expose the use of a product based on collagen for preventing immunological rejection of an allogeneic origin implant.

The document EP1920788A1 relates to a matrix containing mesenchymal stem cells and platelet-rich plasma, for use in the preparation of implants for the treatment of periodontal or bone pathologies, wherein said matrix may contain collagen, however the present invention does not teach nor suggest that the product contains platelet-rich plasma. On the other hand, this document neither teaches nor suggests the construct with the particular elements of the present invention nor the steps to obtain it.

Additionally, there are scientific publications that describe the use of stem cells in the implant of dental pieces with appropriate matrices, such as Zhou Y. et al. “Periodontal healing by periodontal ligament cell sheets in a teeth replantation model”. Arch. Of Oral Biology 57 (2012) 169-176, and Zhao et al. “The combined use of cell sheet fragments of periodontal ligament stem cells and platelet-rich fibrin granules for avulsed tooth reimplantation”. Biomaterials 34 (2013) 5506-5520; however, none of these documents describe the construct of the present invention with the particular elements described, nor the method to manufacture it, nor the use of allogeneic or heterologous stem cells to create constructs for preventing immunological rejection, as does the present invention.

Therefore, it is necessary to have products useful in transplant, whether allotransplant or xenotransplant and methods for producing them that do not present immunological rejection when they are transplanted, and that do not produce xenovirus infection during the transplant. To solve this technical problem, a construct is presented that prevents the immunological rejection produced when it is used in transplant in the treatment of diseases (without the use of immunosuppressive drugs), either xenotransplant or allotransplant; a method for obtaining said construct; a method for producing collagen in a gel state, either in 3-D form or as dry lyophilized spongy collagen molds with the shape of the element to be transplanted; and finally, the use of the construct for preventing the immunological rejection produced during the transplant thereof.

One of the advantages of the present invention is the independence of the cell source, where this independence brings technical benefits (for example ease in cell culture, without depending on a fixed or defined source) and economic (for example lower requirements for maintenance of cell culture compared to obtaining cells from the same patient)

### DESCRIPTION OF THE FIGURES

FIG. 1. It is shown the results of the optimization of the culture medium used in cultures of periodontal ligament stem cells. In the present application was selected the α-MEM medium, standard for stem cell culture, because the doubling time is optimal reducing the time to less than half for this cell type compared to the D-MEM medium high in glucose.

FIG. 2. It is shown the results of the characterization of dental stem cells by flow cytometry. According to the results obtained, it was established that it is advisable to use frozen dental stem cells up to the seventh passage to perform the constructs of the present invention with the maximum potential of the cells. The figure shows that markers for stem cells remain present as expected for CD90, CD29 and STRO-1; while that always absent for CD34. They are also negative for CD-14 (not shown).

FIG. 3. It is shown the microbiological control of tissue for obtaining the collagen product, by means of the methodology described in the present application. It corresponds to CFU/plate in pig tissue treated with povidone-iodine or peracetic acid. Several disinfectants used in the literature were tested and compared with the procedure described in the present invention (povidone-iodine); the results in triplicate showed that povidone-iodine was the only 100% effective for disinfection of pig-skin, prior to submitting it to extraction. The graph shows the comparison with peracetic acid, as an example. Abbreviations: PY—povidone-iodine, PA—Peracetic Acid, CFU—colony forming units, CC—work bell control.

FIG. 4. It is shown results of microbiological control. The photographs of the culture plates show the initial growth of bacterial colonies at the beginning (time 0), and the decrease of CFU at 5 minutes, 30 minutes, 1 hour and 3 hours of exposure to the disinfectant; in the top panel povidone-iodine (PY) and in the lower panel peracetic acid (PA). The povidone-iodine was more effective and completely eliminates the formation of bacterial colonies at 3 hours, while with the peracetic acid the presence of a colony still persists.

FIG. 5. It is shown photographs of stem cell cultures obtained from periodontal ligament, cultured in (a) α-MEM medium and (B) cultured in the same medium on the collagen product (specifically the 3-D matrix), obtained according to the present invention.

FIG. 6. It is shown the SDS-Page gel, the electrophoretic migration of the collagen extract at concentrations of 3.0 μg, 15.0 μg and 30 μg obtained from the analysis of the components of the collagen product obtained from the pig-skin with the method described herein. The typical collagen pattern is observed, in which the α1, α2 and β chains are distinguished; and more clearly with greater concentration of the extract between 15 μg and 30 μg total.

FIG. 7. It is shown the histological section of the 3-D matrix containing the human dental ligament stem cells growing in the collagen 3-D matrix, showing their nuclei stained with the DAPI fluorescent compound. The product of collagen produced to maintain periodontal ligament stem cells in culture is observed. The presence of the cells in the fixed matrix, included in paraffin and cut with a microtome, is shown by immunofluorescence of the nuclei with DAPI staining (40×). The nuclei distributed throughout the matrix are observed, showing that the cells are integrated into the collagen mesh. This product with cells is used to cover the element to be transplanted.

FIG. 8. It is shown an example of the human bio-tooth with regenerated periodontal ligament strongly adhered to the tooth matrix, in such a way that it is possible to hold it with a clamp when extracting it from the back of the mouse (figure a); in figure B) and C) of the center and bottom are histological sections of this tooth after demineralization, wherein the structure of the dentin tubules of the dentine (D) transversally and sagittally cut, with a clear presence of cement (C) and fibers of the periodontal ligament (PDL) in close association with the surface of the dentin. On the other hand, the labeling with antibodies against human mitochondria clearly confirms the presence of human cells differentiated from human stem cells in the construct implanted in mouse without immunosuppression.

### DESCRIPTION OF THE INVENTION

In a first aspect, the present invention relates to a construct for transplant, either xenotransplant or allotransplant, which does not generate immunological rejection, comprising:
a) cells with regenerative capacity;
b) collagen in a 3-D gel state, or also called a collagen 3-D matrix, obtained from pig's head cheeks;
c) culture medium;
d) an element aimed to transplant; and
e) dry lyophilized spongy collagen molded with the shape of the element to be transplanted.

The construct is shaped so that the cells with regenerative capacity (a) are cultured on the collagen in the 3-D gel state (b), and the culture medium (c) is on the cells with regenerative capacity (a); said culture medium with cells and collagen in the 3-D gel state is wrapping the element to be transplanted (d); the element to be transplanted wrapped with cells and the 3-D collagen is inside the dry lyophilized spongy molded collagen (e), in such a way that the resulting product is immersed and surrounded by a second layer of dry lyophilized collagen.

In a specific embodiment, cells with regenerative capacity (a) are selected from the group comprising: allogeneic or autologous stem cells, or induced stem cells or progenitor cells from cell lines leading to specific tissues, or tissue fragments from organs maintained alive or in development. More specifically, the stem cells are cells that are selected from the group comprising: mesenchymal cells of adipose origin, bone marrow, umbilical cord, dental pulp, dental papilla, dental follicle, periodontal ligament, buccal epithelium, condylar ligament, cardiac muscle, covering epithelia, maxillofacial or cranial flat bones, osseous progenitor cells, endothelials. These stem cells are allogeneic or autologous.

In a specific embodiment, the element to be transplanted (d) is selected from the group comprising: tooth, cranial bone, maxilla facial bone, iliac bone, long bones, developing dental organ, kidney, liver, heart, tracheal tube, lung, cornea, motor nerves, ligaments, cartilage, skin. Specifically, the element to be transplanted is human adult mineralized tooth. In case it is a tooth, the construct for transplant can be called bio-tooth.

In a second aspect of the present invention, it is described a method for producing the construct for transplant, either xenotransplant or allotransplant, which does not generate immunological rejection, which comprises the steps of:
a) preparing an element aimed to be transplanted fresh at room temperature between 20 to 25° C., or previously kept cold between 4° C. to 10° C. or frozen (−20° C. to −170° C. in cryopreservation);
b) coating culture plates with collagen in the 3-D gel state in the form of a sheet on the plate, then depositing the cells with regenerative capacity, such as allogeneic or autologous stem cells, or induced stem cells or progenitor cells of cell lines conducive to specific tissues, or tissue fragments of organs maintained alive or in development, and covering with the culture medium according to the cell type and incubating until evidencing by microscopic observation the incorporation of the cells to the three-dimensional matrix from 4 hours to 24 hours;
c) disposing of dry lyophilized spongy collagen molded with the shape of the element to be transplanted;
d) take the culture sheet with stem cells from step (b), removing the excess of culture medium by contact on absorbent paper and rolling it completely on the element to be transplanted;
e) covering or immersing the construct obtained in step d), which consists of the element to be transplanted incorporated into the matrix 3-D collagen, in the collagen molds of step (c); in such a way that the three-dimensional collagen 3-D gel-like construct is immersed and surrounded by a second layer of dry lyophilized spongy collagen (spongy mold type with or without crosslinking, described in the specification for the tooth) and molded according to the shape of the implant and
f) storing frozen at a temperature between −80° C. to −150° C. until its use.

In a specific embodiment, the element to be transplanted from step a) is selected from the group comprising: tooth, cranial bone, maxilla facial bone, iliac bone, long bones, developing dental organ, kidney, liver, heart, tracheal tube, lung, cornea, motor nerves, ligaments, cartilage, skin. Specifically, the element to be transplanted is human adult mineralized tooth. In case it is a tooth, the construct for transplant can be called bio-tooth.

In a particular embodiment, in the case that the tissue to be transplanted is a tooth, the following method is considered to treat the tooth prior to step a):
i) decellularize, by rinsing with ultrafiltered water, immersed in a solution of 17% EDTA between 5 to 15 minutes at neutral pH (7.0 to 7.2) at room temperature (20 to 25° C.) and then maintained between 30 seconds to 5 minutes in a solution of between 10 to 30% citric acid and washed with ultra-pure water.
ii) disinfect, immersing in povidone-iodine for 30 to 90 minutes with agitation, rinse and disinfect with a solution of NaOH at pH 14 in buffer for a period of time between 5 to 15 minutes to finally rinse in ultra-pure water to reach a pH between 5 to 9 and maintain in saline solution or sterile phosphate buffer saline (PBS), in cold, or in 70% ethanol, or maintain dry in a sterile container until its use.

Additionally, if the root tips are closed, they can be opened to perform root and chamber cleaning using conventional endodontic techniques with a type H or K lime, prior to decellularization, leaving the tooth with its channel and root free of cell, maitaining the mineral and organic part.

In another specific embodiment, the stem cells of step b) are stem cells that are selected from the group comprising: mesenchymal cells of adipose origin, bone marrow, umbilical cord, dental pulp, dental papilla, dental follicle, periodontal ligament, buccal epithelium, condylar ligament, cardiac muscle, covering epithelia, maxillofacial or cranial flat bones, bone progenitor cells, endothelial. These stem cells are allogeneic or autologous.

These stem cells are cultured under the following conditions:

Extracting periodontal ligament from the lower third of a tooth, being able to be an erupted third molar or any erupted tooth, obtained from the patient or donor, or from an animal dental piece (pig, dog or other); it is crushed in saline solution (PBS), transferred to dishes covered with a collagen 3-D matrix and culture medium α-MEM is added with FBS (10% to 15%) or with autologous human serum (10% to 15%) to obtain the stem cells. Once the confluence of 60% to 75% is reached, it is digested with collagenic enzymes, and then expanded in α-MEM medium with FBS or autologous human serum, and can be used for implants from the first expansion to the seventh passage, fresh or after freezing and unfreezing; during cultivation, the cell selection process allows to obtain stem cells characterized by flow cytometry or a mixture of stem cells with other types of cells in differentiation pathways.

Alternatively, prior to use, the cells can be obtained directly in culture dishes without collagen by enzymatic extraction and culture in α-MEM medium with 10% or 15% FBS or with 10% to 15% autologous human serum until they reach an optimal confluence (60% to 75%) to expand and freeze until its use. At the time of use they are grown on the collagen 3-D matrix as described above and the sheet with cells is used to completely roll over the element to be transplanted from step a) of the method for producing a construct for transplant.

In addition, prior to covering the tooth with the collagen 3-D matrix and stem cells, the pulp cavity of the mineralized and decellularized matrix of the tooth to be transplanted is filled with dental pulp stem cells (DPSCs) from erupted teeth and extracted by orthodontic indication or the cells of the apex of a third molar in development (SCAPs) or the DPSCs of this type of tooth, allogenic or autologous grown and maintained by the expanding standard method of mesenchymal stem cell of dental origin using preferably the α-MEM medium in the collagen 3-D matrix described herein, with or without VEG (blood vessel growth factor) or alternatively, instead of dental stem cells, cells in differentiation pathways are introduced by a short induction in a standard medium for odontoblastic differentiation that preferably contains the culture medium α-MEM, supplemented with 10% FBS (or autologous serum) together with ascorbic acid, glycerol phosphate. This procedure is performed with the help of a syringe in such a way that the collagen gel containing the cells completely fills the pulp cavity; then, proceed as described in this section from (d) onwards to complete the construct with the gel sheet containing periodontal ligament stem cells and the spongy mold that will allow the dental implant in the alveolar niche.

In case the element to be transplanted is maxillofacial bone, it undergoes the following steps:
i) clean attached tissues, immersion in buffer Tris-NaCl or PBS between 30 seconds to 5 minutes or 70% ethanol between 1 and 5 minutes, then rinse in ultra-pure water to coat the collagen 3-D matrix seeded with mesenchymal stem cells or precursor cells;
ii) alternatively, after the cleaning described in (I), it is subjected to decellularization following the same steps as the tooth.

Decellularization is carried out applying the same principles previously described for the mineralized matrix of a dental piece, (except for the endodontic process) a diluted acid (for example hydrochloric acid) is used for 15 minutes or more, depending on the size of the bone fragment, and it is decellularized by a range of 15 minutes at several hours (3 to 8 hours depending on the size of the bone fragment) with acetone or a diluted nitric acid solution. Finally, it is rinsed in sterile ultrapure water for 24 hours and immersed in ethanol between 60% and 90% until its use.

The decellularized bone is coated with the mixture of 3-D matrix of collagen-mesenchymal stem cells or osteoblastic precursors, containing the standard α-MEM culture medium supplemented with 10% FBS, dexamethasone, ascorbic acid, glycerophosphate. These elements are inserted in a collagen mold subjected to crosslinking as previously described and transplanted into the body without immunosuppression.

In a third aspect of the present invention, it is described a method for producing the collagen in the gel state, either in the 3-D gel state where the cells with regenerative capacity are cultivated or as dry lyophilized spongy collagen molds with the form of the element to be transplanted, which includes:
a) disinfecting a segment of pig-skin (specifically cheek pig's head) immersing it in a povidone-iodine commercial solution in a concentration between 1% and 30%, for a period of 3 to 6 hours, and then brushing vigorously on both sides and rinse with ultra-pure quality water;
b) extract and crush the dermis of the skin segment obtained from step a), and subsequently mixing with a solution between 1 and 10 M of sodium acetate, and the pH is raised to a range from 10 to 14 with a solution hydroxide sodium 10 to 15 M for a period of time between 30 minutes and 2 hours at room temperature (20 to 25° C.) to sterilize the solution and extract the proteins;
c) washing the mixture resulting from step b) 2 to 7 times with a solution of sodium acetate between 1 M and 10 M, collecting the proteins by centrifugation at 9,000 or more rpm, in cold between 0 and 10° C.; followed by 2 to 7 washes with ultra-pure quality water by the same centrifugation method; then the sediment is solubilized in a solution of dilute acetic acid between 0.01N and 0.5N in a ratio of 1 liter per 1.5 g of the solid, by stirring between 0 and 10° C.;
d) recovering by centrifugation between 0 and 10° C. at 9,000 or more rpm, and then separating the suspended solid particles from the solution by gauze filtration; subsequently, the solution is frozen (−20° C. to −30° C.) and subjected to a lyophilization process to remove the water; and
e) maintain frozen the lyophilization product until its use, obtaining the collagen in gel form.

Alternatively, the disinfected pig-skin obtained in step a) is frozen between −80° C. to −196° C. to maintain it stored until its use.

### Method for Preparing Collagen Molds

Additionally, the collagen obtained by the method described above from steps a) to e) can be presented in the form of dry lyophilized spongy molds for rolling the elements to be transplanted following the additional steps of:
f) measuring the mass of the lyophilized of step e) and dissolve in acetic acid (0.5N or higher concentration) at 4° C. (±1.0° C.), until obtaining a concentration of 20 to 30 mg/mL;
g) introducing the dissolved product from step f) into sterile plastics molds according to the size and shape of the implant,
h) freezing at temperature between −20 to −24° C. at least 24 hours;
i) lyophilizing the frozen product from step h) and
j) detaching the product from the plastic molds using a spatula, storing it in a dry place at room temperature (20 to 25° C.) until its use (it can be stored for at least 12 months).

The molds of step g) can be thin or thick sheets, or cones of varying thickness, or tubules of varying thickness, or another form as appropriate to the tissue to be transplanted.

Additionally, the spongy collagen molds obtained according to steps f) to j) are treated by proper methods of cross-linking with non-toxic (innocuous) material, controlling the density of the molded collagen, through the following steps:
i) immersing and incubating the collagen (spongy molds) at room temperature (20° C. to 25° C.) in a solution based on 2.3% w/v N-hydroxysuccinimid (NHS) and 3.83% w/v N-(3Dimethylaminopropyl)-N′-ethylcarbodiimide hydrochloride) (EDC) in a volume between 1 and 40 mL of 70% ethanol, and subsequently perform washes of this collagen in ultra-pure sterile water;
ii) freezing collagen at a temperature between −50 and −5° C. between 12 and 36 hours and then lyophilizing in the same conditions as above, which will be called as spongy type molds by its new consistency, firmer than the gel of the step e).

### Method for Preparing Collagen 3-D Matrix

Optionally, the collagen obtained after step e), can be presented as a collagen 3-D matrix in a gel state for cell culture with the following additional steps:
i) measuring the mass of the lyophilized product obtained from step e) and dissolve in acetic acid (0.01N to 0.05 N) at cold 4° C. (±1.0° C.) to obtain a final required concentration between 2.0 mg/mL to 5.0 mg/mL;
ii) take the completely dissolved product from step k) and dialyze into chloroform at a concentration between 0.5% and 5% to remove impurities, followed by dialysis in dilute acetic acid (0.01N to 0.05 N) at cold 4° C. (±1.0° C.);
iii) obtaining a 3-D biological matrix of gel-type or gel-like collagen that provides three-dimensional environment to the cells in culture; and
iv) storing the product in cold without freezing at temperature between 4° C. to 10° C.

In a last aspect, the present invention refers to the use of said construct for preventing the immunological rejection produced during the transplant thereof.

In the event that the final construct is a bio-tooth, it is used for any of the 32 human teeth, or for pets or animals with dental roots.

### EXAMPLES OF APPLICATION

### Example 1: Obtaining Pig-Skin Collagen and Manufacturing the Collagen 3-D Matrix

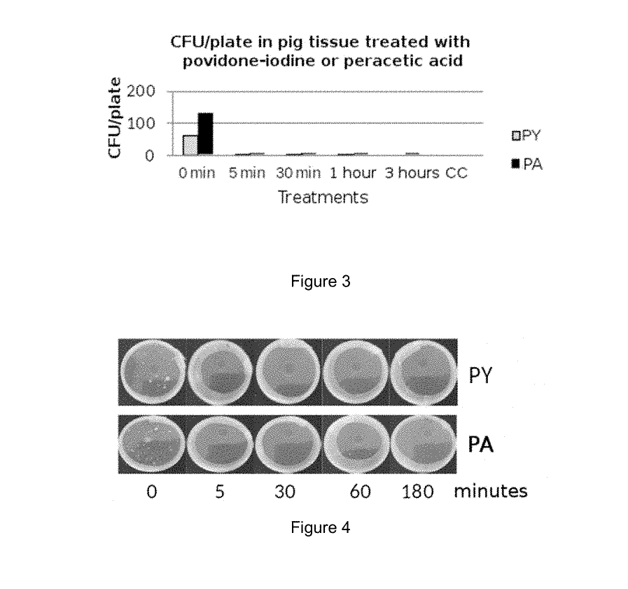

i) Sterilization and freezing of the tissue: Pig's head cheeks skin from 175 to 190 days of age were used. Which was immersed for 3.5 hours in a commercial solution of povidone-iodine 10% (Brand: Centrovet) and brushed on both sides every 30 minutes, in a sterile environment, and rinsed with ultra-pure quality water. The Table 1 and the FIGS. 3 and 4, show the results of analysis of effectiveness of povidone-iodine (PY) used in the present invention compared to the effectiveness of peracetic acid (PA); demonstrating that povidone is more effective, since it eliminates 100% of the microbial growth at 3 hours post treatment and maintains the integrity of the collagen obtained later, while the peracetic acid damages the quality of the collagen by altering the viscosity of the final product, in such a way that it does not acquire the necessary consistency to be used in the implants. The disinfected tissue is bagged and sealed to freeze at −80° C. (±2.0° C.) in ultra freezer (Brand: Thermoline).

TABLE 1
Treatment of pig tissue with disinfectants.

| | UCF/plate in pig tissue treated with PY or PA | | | | |
|---|---|---|---|---|---|
| | Exposition time | | | | |
| Treatment | 0 min | 5 min | 30 min | 1 hour | 3 hours |
| PY | 61 | 1 | 1 | 6 | 0 |
| PA | 132 | 2 | 3 | 7 | 1 |

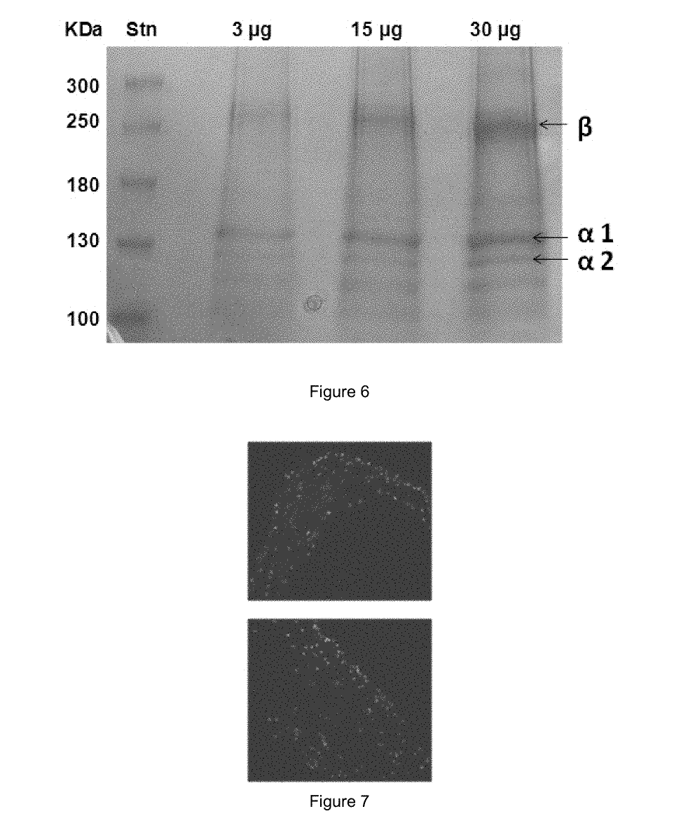

i) Unfreezing and cleaning the tissue: The frozen tissue is placed on a cold surface (4° C.), in a sterile environment to remove adipose and epithelial tissue with the help of a scalpel. The dermis, thus obtained, was cut into pieces of approximately 1 cm², and ground in 200 mL of a solution of 5M sodium acetate (Brand: JT Baker) with a food crusher in short pulses of less than 5 seconds each for preventing raising the temperature. The elimination of possible pathogens in this step was carried out by adjusting the pH of the solution to pH14 with a 12 M NaOH solution for 1 hour and 10 minutes at room temperature (20° C. to 25° C.).
ii) Extraction and solubilization of collagen: The mixture of grounded and disinfected tissue was washed 5 times with 5M sterile sodium acetate, by centrifugation at 9500 rpm for 20 minutes in cold in Hettich brand centrifuge at 4° C. Followed by 5 washes with sterile ultra-pure water by the same centrifugation method. The final sediment was extracted in 2 liters of 0.02 N acetic acid for each 3.0-3.2 g of sediment using a stainless steel blender with 45 second pulses under sterile conditions. Followed by cold stirring for 50 hours.
iii) Purification and freezing of Collagen: The obtained collagen solution was cleaned of impurities by centrifugation at 9500 rpm for 20 minutes in Hettich brand centrifuge at 4° C. and 100 mL volumes were filtered in hydrophilic gauze (sterile for therapeutic use) folded in 10 mL folds over sterile bottle. These extracts were frozen at −20° C. for 48 hours to proceed to the lyophilization step.
iv) Lyophilization: Volumes of 100 mL of frozen extract were lyophilized at −85° C. with constant pressure in Labconco brand lyophilizer for approximately 48 hours until dryness of the samples.
v) Obtaining collagen 3-D matrix: It was measured the mass of the product obtained (dry collagen) and dissolved in a proportion of 90 mg per 30 mL of 0.02 N sterile acetic acid, at 4° C. with occasional stirring until its complete dissolution. This solution was introduced into dialysis membranes (SnakeSkin Dialysis Tubing, Brand: Thermoscientific) and dialysed against 1% chloroform for one hour and then, against 0.02 N acetic acid making changes of the solution until the chloroform was eliminated. The obtained gel was removed from the dialysis bag and stored at 4° C. in a sterile package.
vi) Sterility test and analysis: aliquots of the collagen obtained in gel form were taken and incubated at 37° C. in the incubator with 5% CO₂ for 48 hours to monitor its sterility. Its protein composition was also analyzed by electrophoresis in SDS-Page polyacrylamide gels; which showed the typical type-1 collagen pattern. In FIG. 6, at a concentration of 3.0 mg, the α1, α2 and β chains are distinguished; those that are observed with greater clarity when increasing the amount to 15 mg and 30 mg of the extract of collagen obtained.
vii) The collagen obtained at a concentration of 3.0 mg/mL (+/−0.1 mg, approximately) was used to cover cell culture plates and to mix with cell solution obtained from fresh dental or bone tissues; this collagen gives a three-dimensional environment (3-D) to the cells in culture.

### Example 2: Method for Manufacturing the Dry Lyophilized Spongy Collagen Molds

The spongy collagen matrix used for transplants of the bio-tooth or bone, or allogeneic tooth germs was prepared from the lyophilized collagen obtained according to the procedure in example 1 to point (v). It was measured the mass of a minimum of 200 mg of the dry collagen, it was dissolved in a solution of 10 mL 0.5 N acetic acid and it was introduced with the help of a spatula to sterile plastic molds to then, once solidified, introduce the element to be transplanted. The mold for teeth construct has the shape of a cylinder covered by one of its ends with walls of 2 mm thick and a depth of approximately 2 cm, in such a way that the tooth to be transplanted was completely inserted inside it.

These molds were frozen at −20° C. for at least 48 hours and then subjected to the water sublimation process by lyophilization in a labconco brande lyophilizer.

The crosslinking of the collagen fibers of these molds was carried out for six hours in 70% ethanol solution containing 0.230 g of NHS and 0.383 g of EDC at room temperature (20° C.-25° C.).

Then, these molds were rinsed in ultra-pure water for six hours, frozen at −20° C. and subjected to the lyophilization process in the same conditions previously described here.

### Example 3: Method for Manufacturing the Bio-Tooth or Construct to be Transplanted

Matrices of human teeth (premolar of young people under 21 years of age) were obtained, decellularized and cleaned as described in the present application, the pulp chamber and roots were cleaned, treated for 10 minutes with 17% EDTA and 1 minute with citric acid to then disinfect with povidone-iodine and NaOH and finally, after rinsing they were maintained in 70% ethanol until its use.

It was performed a periodontal ligament stem cells culture as previously described, specifically it was used disposable plates 60 mm diameter Nunc brand and the α-MEM culture medium (Invitrogen) with 15% fetal bovine serum (FBS, Invitrogen), on a layer of 3-D collagen prepared according to our method described herein. It was expected to reach confluence of approximately 70%, the ligament tissue residues were removed and the cells were extracted from the collagen by treatment with 3.0 mg/mL type-1 collagenase (Invitrogen) for 5 minutes at 37° C., dispersing with pipette several times, and collecting by centrifugation, 3 times. The culture was expanded in a 1:3 ratio, in 100 mm dishes covered with the collagen 3-D matrix.

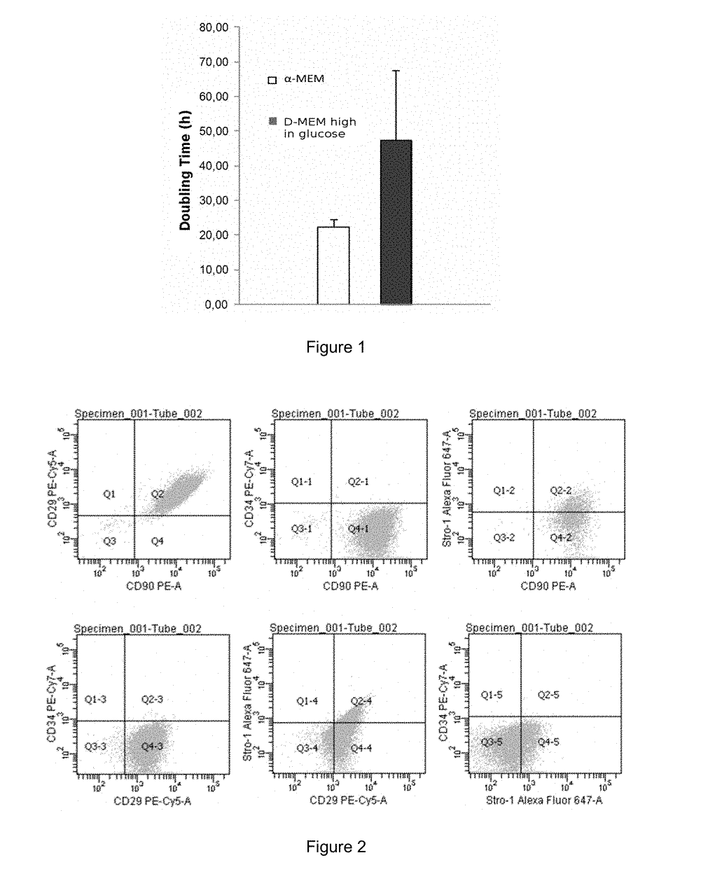

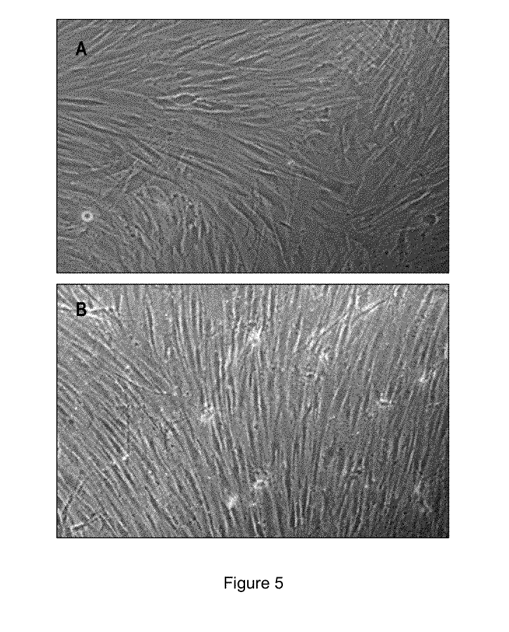

FIG. 1 shows the results of the optimization of the culture medium used in our periodontal ligament stem cells cultures, therefore, the cultured cells were used, frozen and expanded until the fifth transfer in this medium. FIG. 2 shows the results of flow cytometry for cells grown under these conditions. Example of expansion of the stem cells of the periodontal ligament cultured in α-MEM medium directly on culture dishes is shown in FIG. 5(A); whereas in FIG. 5(B) an example of the expansion of these cells in collagen 3-D medium using the same medium is shown. FIG. 7 shows histological section of the 3-D matrix containing the human dental ligament stem cells growing in the collagen 3-D matrix, showing their nuclei stained with the DAPI fluorescent compound.

Once the approximate confluence of 70% was obtained, the collagen 3-D sheet containing the periodontal ligament stem cells was taken and wound onto the root section of the decellularized dental matrix, prepared and dried in a sterile environment for 5 minutes (i), building a three-dimensional matrix of five layers (at least) of cells on this dental matrix.

### Example 4: Use of the Bio-Tooth, Results Obtained

The bio-tooth constructed with dental matrices of human teeth and the 3-D collagen containing the periodontal ligament stem cells was introduced into a mold of cross-linked collagen as described herein and transplanted under anesthesia in vivo to mice without immunosuppression, in regions such as, intraperitoneal or on the back under the skin to incubate for 2 months. An implant per animal was made by completing a total of twenty implants with the constructs of human bio-teeth with collagen as described here, without the use of immunosuppression. The control animals received implants of human constructs with stem cells without collagen. The animal use and care protocols were approved by the Committee Institutional center ADG I+D, the surgeries were performed by a veterinarian and researcher with more than 30 years of experience in experimental studies in laboratory rodents. The animals treated with analgesics were left with water and food ad libitum and were monitored the first hours to ensure their well-being; then, with daily observation, and routine care until the moment they are sacrificed without pain by cervical dislocation.

Then, the constructs were extracted by means of a clamp. They were fixed in 4% formaldehyde in PBS buffer for one week with low shaking. The constructs were demineralized in 10% EDTA until obtaining a soft consistency to the touch with clamps and were included in paraffin for the routine histology process with microtome cuts. The 20 transplants of bio-teeth constructed and implanted with collagen molds that were maintained in animals without immunosuppression showed growth of the periodontal ligament highly adhered to the dental root matrix, whereas the control constructs without collagen showed no tissue development in its matrix showing destruction and loss of human stem cells.

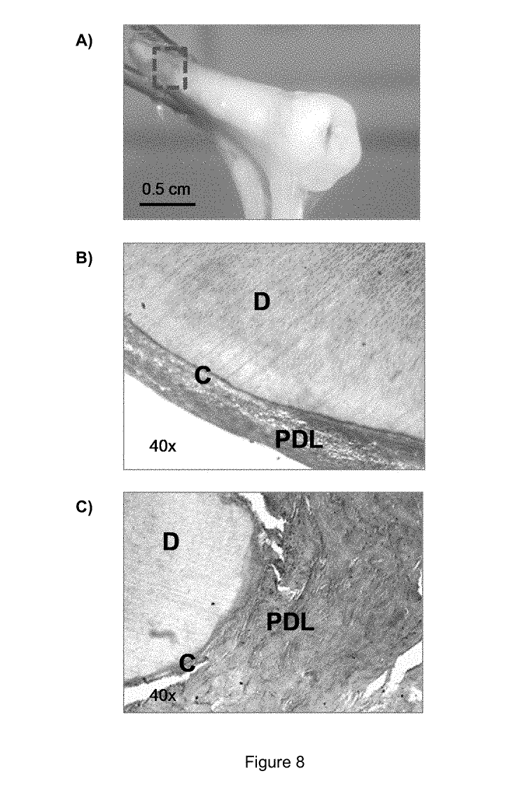

FIG. 8 shows an example of the human bio-tooth with regenerated periodontal ligament strongly adhered to the tooth matrix, in such a way that it is possible to hold it with a clamp when extracting it from the back of the mouse (top photograph); In the photograph of the center and bottom, histological sections of this tooth are shown after demineralization, where the structure of the dentin tubules of the dentin (D) transversally and sagittally cut is observed, with a clear presence of cement (C) and fibers of the periodontal ligament (PDL) in close association with the surface of the dentin. On the other hand, the labeling with antibodies against human mitochondria clearly confirms the presence of human cells differentiated from human stem cells in the construct implanted in mice without immunosuppression.

1. A construct for transplant, either xenotransplant or allotransplant, which does not generate immunological rejection, comprising:
a) cells with regenerative capacity;
b) collagen in 3-D gel state, or also called collagen 3-D matrix, obtained from pig's head cheeks;
c) culture medium;
d) an element aimed to be transplanted; and
e) dry lyophilized spongy molded collagen with the shape of the element to be transplanted.

2. The construct according to claim 1, wherein the construct is shaped so that the cells with regenerative capacity (a) are cultured on the collagen in the 3-D gel state (b), and the culture medium (c) is on the cells with regenerative capacity (a); said culture medium with cells and collagen in the 3-D gel state is wrapping the element to be transplanted (d); the element to be transplanted wrapped with cells and the 3-D collagen is inside the dry lyophilized spongy molded collagen (e), in such a way that the resulting product is immersed and surrounded by a second layer of dry lyophilized collagen.

3. The construct according to claim 1, wherein the cells with regenerative capacity (a) are allogeneic or autologous stem cells, or induced stem cells or progenitor cells of cell lines leading to specific tissues, or tissue fragments of organs maintained alive or in development.

4. The construct according to claim 3, wherein the stem cells are allogeneic or autologous cells that are selected from the group consisting of: mesenchymal cells of adipose origin, bone marrow, umbilical cord, dental pulp, dental papilla, dental follicle, periodontal ligament, buccal epithelium, condylar ligament, cardiac muscle, covering epithelia, maxillofacial or cranial flat bones, bone progenitor cells, and endothelial.

5. The construct according to claim 1, wherein the element to be transplanted (d) is selected from the group consisting of: tooth, cranial bone, maxilla facial bone, iliac bone, long bones, developing dental organ, kidney, liver, heart, tracheal tube, lung, cornea, motor nerves, ligaments, cartilage, and skin.

6. A method for producing a construct for transplant, either xenotransplant or allotransplant, which does not generate immunological rejection, wherein it comprises the steps of:
a) preparing an element aimed to be transplanted fresh at room temperature between 20 to 25° C., or previously maintained cold between 4° C. to 10° C. or frozen at a temperature between −20° C. to −170° C. in cryopreservation;
b) coating culture plates with the collagen in the 3-D gel state in the form of a sheet on the plate, then depositing the cells with regenerative capacity, such as, allogeneic or autologous stem cells, or induced stem cells or progenitor cells of cell lines conducive to specific tissues, or tissue fragments of organs maintained alive or in development, and cover with the culture medium according to the cell type and incubate until evidencing by microscopic observation the incorporation of the cells to the three-dimensional matrix from 4 hours to 24 hours;
c) disposing of dry lyophilized spongy collagen molded with the shape of the element to be transplanted;
d) taking the culture sheet with stem cells from step (b), removing the excess of culture medium by contact on absorbent paper and rolling it completely over the element to be transplanted;
e) covering or immersing the construct obtained in step d), which consists of the element to be transplanted incorporated in the collagen 3-D matrix, in the collagen molds of step (c); in such a way that the three-dimensional 3-D collagen gel-like construct is immersed and surrounded by a second layer of dry lyophilized spongy collagen and molded according to the shape of the implant; and
f) storing frozen at a temperature between −80° C. to −150° C. until its use.

7. The method according to claim 6, wherein the element to be transplanted from step a) is selected from the group consisting of: tooth, cranial bone, maxilla facial bone, iliac bone, long bones, developing dental organ, kidney, liver, heart, tracheal tube, lung, cornea, motor nerves, ligaments, cartilage, and skin.

8. (canceled)

9. The method according to claim 6, wherein prior to step a), the human mineralized tooth is treated as follows:
i) decellularizing, by rinsing with ultrafiltered water, immersed in a solution of 17% EDTA between 5 to 15 minutes at neutral pH between 7.0 to 7.2 at room temperature between 20 to 25° C. and then maintained between 30 seconds to 5 minutes in a solution of between 10 to 30% citric acid and washed with ultra-pure water;
ii) disinfecting, immersing in povidone-iodine for to 90 minutes with stirring, rinse and disinfect with a solution of NaOH at pH 14 in buffer for a period of time between 5 to 15 minutes to finally rinse in ultra-pure water until reaching a pH between 5 to 9 and maintain in saline or sterile phosphate buffered saline (PBS), in cold, or in 70% ethanol, or maintain dry in a sterile container until its use.

10. The method according to claim 6, wherein the stem cells of step b) are allogeneic or autologous stem cells which are selected from the group consisting of: mesenchymal cells of adipose origin, bone marrow, umbilical cord, dental pulp, dental papilla, dental follicle, periodontal ligament, buccal epithelium, condylar ligament, cardiac muscle, covering epithelia, maxillofacial or cranial flat bones, bone progenitor cells, and endothelial.

11. The method according to claim 10, wherein the stem cells are cultured under the following conditions:
i. extracting the periodontal ligament from the lower third of a tooth, which can be an erupted third molar or any erupted tooth, or an animal tooth; crush in saline (PBS), transfer to dishes covered in a collagen 3-D matrix and add α-MEM culture medium with FBS in a concentration of 10% to 15% or with autologous human serum in the same concentration for obtain the stem cells; and
ii. digesting with collagenic enzymes once reached the confluence of 60% to 75%, and then expand in α-MEM medium with FBS or with autologous human serum.

12. (canceled)

13. The method according to claim 6, wherein the maxillofacial bone is subjected to the steps of:
i) cleaning attached tissues, immersion in Tris-NaCl buffer or PBS between 30 seconds at 5 minutes or in 70% ethanol between 1 and 5 minutes, and then rinse in ultra-pure water to coat the collagen 3-D matrix seeded with mesenchymal stem cells or precursor cells;
ii) alternatively, after the cleaning described in (i) subjecting to decellularization.

14. A method for producing the collagen in gel state, wherein comprising the following steps:
a) disinfecting a segment of pig's head cheek;
b) extracting and crushing the dermis of the skin segment obtained
from step a), and subsequently mixing with a solution between 1 and 10 M of sodium acetate, and the pH is raised to a range from 10 to 14 with a solution hydroxide sodium;
c) washing the mixture resulting from step b) 2 to 7 times with a solution of sodium acetate; followed by 2 to 7 washes with ultra-pure quality water; then the sediment is solubilized in a solution of dilute acetic acid;
d) recovering by centrifugation between 0 and 10° C. at 9,000 or more rpm, and then separating the suspended solid particles from the solution; subsequently, the solution is frozen and subjected to a lyophilization process; and
e) maintaining frozen the lyophilization product until use, obtaining the collagen in gel form.

15. The method according to claim 14, wherein the disinfection of the pig-skin segment of step a) is carried out by immersing it in a commercial povidone-iodine solution in a concentration between 1% and 30%, for a period of 3 to 6 hours, and then brush vigorously on both sides and rinse with ultra-pure quality water.

16. The method according to claim 14, wherein alternatively, the disinfected pig-skin obtained in step a) is frozen between −80° C. to −196° C. to maintain it stored until its use.

17. The method according to claim 14, wherein in step b), the sodium hydroxide solution is in a concentration between 10 to 15 M and is maintained in contact with the mixture for a period of time between 30 minutes and 2 hours at room temperature 20° C. to 25° C. to sterilize the solution and extract the proteins.

18. The method according to claim 14, wherein in step c), the first washing is carried out with sodium acetate between 1M and 10M, collecting the proteins by centrifugation at 9,000 or more rpm in cold between 0 and 10° C.

19. The method according to claim 14, wherein in step c), after the second washing, the sediment is solubilized in an acetic acid solution diluted between 0.01N and 0.5N in a proportion of 1 liter per 1.5 g of the solid, by stirring between 0 and 10° C.

20. The method according to claim 14, wherein in step d), the separation of the solid particles in suspension is carried out by gauze filtration.

21. The method according to claim 14, wherein the collagen obtained in the form of a gel in step e) is in the form of dry lyophilized spongy molds for rolling the elements to be transplanted following the additional steps of:
f) measuring the mass of the lyophilisated from step e) and dissolving in acetic acid with a concentration greater than or equal to 0.5N at 4° C.±1.0° C., until obtaining a concentration of 20 to 30 mg/mL;
g) introducing the dissolved product from step f) into sterile plastic molds according to the size and shape of the implant,
h) freezing at a temperature between −20° C. to −24° C. for at least 24 hours;
i) lyophilizating the frozen product of step h) and
j) detaching the product from the plastic molds using a spatula to be stored in a dry place at room temperature between 20 to 25° C. until its use.

22. The method according to claim 21, wherein the molds of step g) can be thin or thick sheets, or cones of varying thickness, or tubules of varying thickness, or another shape as appropriate to the tissue to be transplanted.

23. The method according to claim 21, wherein the spongy collagen molds obtained according to steps f) to j) are treated by proper methods of cross-linking with non-toxic (innocuous) material, controlling the density of the molded collagen, through the following steps:
i. immersing and incubating the spongy molded collagen at room temperature between 20° C. to 25° C. in a solution based on 2.3% w/v N-hydroxysuccinimide (NHS) and 3.83% w/v N-(3Dimethylaminopropyl)-N′-ethylcarbodiimide hydrochloride) (EDC) in a volume between 1 and 40 mL of 70% ethanol, and subsequently perform washings of this collagen in sterile ultrapure water;
ii. freezing the collagen at a temperature between −50 and −5° C. between 12 and 36 hours and then lyophilize under the same conditions as before.

24. The method according to claim 14, wherein the collagen obtained after step e), is presented as a collagen in the 3-D gel state, or 3-D matrix, for the cultivation of cells with the following additional steps:
i) measuring the mass of the lyophilized product obtained from step e) and dissolving in acetic acid at a concentration between 0.01N to 0.05N cold at 4° C.±1.0° C. until obtaining a required final concentration between 2.0 mg/mL a 5.0 mg/mL;
ii) take the completely dissolved product from step i) and dialyze into chloroform at a concentration between 0.5% and 5% to remove impurities, followed by dialysis in dilute acetic acid at a concentration between 0.01N to 0.05 N cold at 4° C.±1.0° C.;
iii) obtaining a 3-D biological matrix of gel-type collagen or gel-state that provides three-dimensional environment to the cells in culture; and
iv) storing the product in cold without freezing at temperature between 4° C. to 10° C.
